(19) United States
(12) Patent Application Publication
(10) Pub. No.: US 2023/0329744 A1
(43) Pub. Date: Oct. 19, 2023

(54) PUNCH ASSEMBLY TO FACILITATE HAIR FOLLICLE REMOVAL

(71) Applicant: Cole Isolation Technique, LLC, Alpharetta, GA (US)

(72) Inventors: John P. Cole, Alpharetta, GA (US); Tesfaye Habtemariam, Alpharetta, GA (US)

(21) Appl. No.: 17/720,949

(22) Filed: Apr. 14, 2022

(51) Int. Cl. A61B 17/3205; A61B 17/34; A61F 2/10

(52) U.S. Cl. CPC A61B 17/32053; A61B 17/3468; A61F 2/10; A61B 2017/00752

(57) ABSTRACT

Briefly, a punch assembly, such as for removing hair follicles from surrounding tissue, along with methods utilizing the punch assembly, are disclosed. A punch assembly may include a substantially cylindrical body having a proximal end and a distal end, the substantially cylindrical body being formed about a central axis and extending distally from the proximal end. The substantially cylindrical body may include a hair follicle-receiving chamber at least partially defined by an interior wall within the substantially cylindrical body, the interior wall extending distally from the proximal end. The punch assembly may additionally include denticulations arranged at the distal end of the substantially cylindrical body. The denticulations may be beveled in a direction away from the central axis so as to form an outer cutting edge.

### BACKGROUND

### 1. Field

The present disclosure relates generally to surgical tools and, more particularly, to surgical tools to facilitate removal of a hair follicle from a first region of the scalp to a second region of the scalp.

### 2. Information

As a treatment to mitigate hair loss at the human scalp, tools have been developed to remove a hair follicle from a first region of the human scalp, such as a region at which hair actively grows, to a second region of the human scalp, at which hair does not actively grow. Such tools may enable methods that permit removal/replacement of thousands of hair follicles, which may assist in making an aging individual appear younger and more vibrant.

However, relocation of hair follicles from a first region of the scalp to a second region of the scalp may represent a time-consuming process as hair follicles are extracted from the first region and implanted into the second region. In addition, in view of removal and replacement of large numbers of hair follicles, a premium may be placed on surgical tools and processes that reduce the time needed to complete the healing process so that the individual may quickly assume normal day-to-day activities without use of bandages, protective coverings, anti-infection treatments, and so forth. Thus, it may be appreciated that development of approaches to decrease removal/replacement of hair follicles as well as approaches toward decreasing healing time of affected regions of the human scalp continues to be an active area of investigation.

### SUMMARY

One general aspect includes a punch assembly for removing hair follicles from surrounding tissue. The punch assembly including a substantially cylindrical body having a proximal end and a distal end, the substantially cylindrical body formed about a central axis and extending distally from the proximal end, the substantially cylindrical body having a hair follicle-receiving chamber at least partially defined by an interior wall within the substantially cylindrical body. The interior wall extending distally from the proximal end. The punch assembly also includes a plurality of denticulations arranged at the distal end of the substantially cylindrical body, the plurality of denticulations being beveled in a direction away from the central axis so as to form an outer cutting edge.

In particular embodiments, the punch assembly the number of denticulations includes between 3 and 10 denticulations. In particular embodiments, number of denticulations includes of 5 or 6 denticulations. In particular embodiments, the substantially cylindrical body of the punch assembly includes a narrowed portion that terminates at the distal end, the narrowed portion having a length of between 1.75 mm and 5.0 mm. in particular embodiments, the narrowed portion of the punch assembly comprises a width of between 0.5 mm and 0.75 mm. in particular embodiments, the denticulations are beveled at an angle of between about 15° and about 45° with respect to the central axis. In particular embodiments, the denticulations are beveled at an angle of between about 22° and about 38° with respect to the central axis.

Another general aspect includes a method of removing a hair follicle from surrounding tissue for use with a punch assembly having a distal end and a proximal end. The method may include situating a portion of the distal end of the punch assembly to be in contact with the surrounding tissue so as to substantially encircle the hair follicle. The punch assembly may include a substantially cylindrical body formed about a central axis and extending distally from the proximal end. The method may also include distorting at least a portion of the surrounding tissue by applying a lateral force to the distal end of the punch assembly. The method may also include separating the substantially encircled hair follicle from the surrounding. The method may also include forming, responsive to separating the substantially encircled hair follicle from the surrounding tissue, in which the wound includes a scalloped-shaped inferior margin and an irregularly shaped superior margin.

In particular embodiments, the separating of the substantially encircled hair follicle from the surrounding tissue, which includes tearing the substantially encircled hair follicle from the surrounding tissue. In particular embodiments, the method may include incising into the surrounding tissue and in a direction substantially parallel to the hair follicle while maintaining the situated portion of the distal end of the punch assembly in contact with the surrounding tissue. In particular embodiments, the method may include maintaining the lateral force to the distal end of the punch assembly while incising into the surrounding tissue. In particular embodiments, the method may include extracting the hair follicle in a manner that forms a wound in the surrounding tissue having an ellipse shape with a measure of eccentricity of between about 0.5 and about 0.95. In particular embodiments, the ellipse shape may include a measure of eccentricity of between about 0.63 and about 0.8. In particular embodiments, the ellipse-shaped wound includes a major axis that substantially aligns with one or more of Langer's lines proximate the wound. In particular embodiments, distorting the portion of the surrounding tissue reduces an axial force to initiate an incision into the surrounding tissue by at least about 25%.

Another general aspect includes a method of removing a hair follicle, which includes disposing a portion of a distal end of a cylinder-shaped punch assembly at an inferior margin of a portion of tissue surrounding the hair follicle. The method may additionally include distorting the shape of the portion of the tissue responsive to application of a lateral force to the cylinder-shaped punch assembly. The method may additionally include incising the portion of the tissue surrounding the hair follicle at the inferior margin. The method may additionally include forming an irregularly-shaped separation in the surrounding tissue at a superior margin of the surrounding tissue.

In particular embodiments, distorting the shape of the portion of the surrounding tissue includes: reducing axial force to bring about the incision of the portion of the surrounding tissue at the inferior margin, extracting the hair follicle responsive to applying force that at least approximately aligns with the central axis of the hair follicle, producing an ellipse-shaped wound responsive to extracting the hair follicle and removing the cylinder-shaped punch assembly from the portion of the surrounding tissue. In particular embodiments, the ellipse-shaped wound includes a major access that substantially aligns with one or more of Langer's lines proximate the wound.

### DESCRIPTION OF THE DRAWINGS

Claimed subject matter is particularly pointed out and distinctly claimed in the concluding portion of the specification. However, both as to organization and/or method of operation, together with objects, features, and/or advantages thereof, it may best be understood by reference to the following detailed description if read with the accompanying drawings in which:

FIGS. 1A-1B are side views illustrating various features of a punch assembly to facilitate hair follicle removal, according to an embodiment.

FIG. 2A is a view showing features of a distal portion of a punch assembly to facilitate hair follicle removal, according to an embodiment.

FIG. 2B is a simplified top view showing features of a punch assembly to facilitate hair follicle removal, according to an embodiment.

FIG. 3A is a diagram showing a punch assembly involved in a hair follicle removal process, according to an embodiment.

FIG. 3B is a top view showing a punch assembly involved in a hair follicle removal process, according to an embodiment.

FIG. 4 is a diagram showing a wound imparted by a punch assembly to facilitate hair follicle removal on a human scalp relative to Langer's lines, according to an embodiment.

FIG. 5 is a diagram showing size of a wound imparted by a punch assembly to facilitate hair follicle removal in relation to dimensions of the punch assembly, according to an embodiment.

FIG. 6 shows a conventional cylindrical punch for hair follicle removal and illustrates disadvantages of such conventional cylindrical punches according to implementation

FIG. 7A shows an ellipse-shaped wound in relation to Langer's lines, according to an embodiment.

FIG. 7B shows an ellipse-shaped wound that intersects Langer's lines, according to an embodiment.

FIGS. 8-9 are flowcharts for methods of using a punch assembly to facilitate hair follicle removal, according to embodiments.

Reference is made in the following detailed description to accompanying drawings, which form a part hereof, wherein like numerals may designate like parts throughout that are corresponding and/or analogous. It will be appreciated that the figures have not necessarily been drawn to scale, such as for simplicity and/or clarity of illustration. For example, dimensions of some aspects may be exaggerated relative to others, one or more aspects, properties, etc. may be omitted, such as for ease of discussion, or the like. Further, it is to be understood that other embodiments may be utilized. Furthermore, structural and/or other changes may be made without departing from claimed subject matter. References throughout this specification to “claimed subject matter” refer to subject matter intended to be covered by one or more claims, or any portion thereof, and are not necessarily intended to refer to a complete claim set, to a particular combination of claim sets (e.g., method claims, apparatus claims, etc.), or to a particular claim. It should also be noted that directions and/or references, for example, such as up, down, top, bottom, and so on, may be used to facilitate discussion of drawings and are not intended to restrict application of claimed subject matter. Therefore, the following detailed description is not to be taken to limit claimed subject matter and/or equivalents.

### DETAILED DESCRIPTION

References throughout this specification to one implementation, an implementation, one embodiment, an embodiment, and/or the like means that a particular feature, structure, characteristic, and/or the like described in relation to a particular implementation and/or embodiment is included in at least one implementation and/or embodiment of claimed subject matter. Thus, appearances of such phrases, for example, in various places throughout this specification are not necessarily intended to refer to the same implementation and/or embodiment or to any one particular implementation and/or embodiment. Furthermore, it is to be understood that particular features, structures, characteristics, or the like described are capable of being combined in various ways in one or more implementations and/or embodiments and, therefore, are within intended claim scope. In general, of course, for the specification of a patent application, these and other issues have a potential to vary in a particular context of usage. In other words, throughout the disclosure, particular context of description and/or usage provides helpful guidance regarding reasonable inferences to be drawn; however, likewise, “in this context” in general without further qualification refers at least to the context of the present patent application.

Some example methods, apparatuses, and/or articles of manufacture are disclosed herein, which may be used in whole or in part, to facilitate and/or support one or more operations and/or techniques for utilizing a punch assembly to facilitate hair follicle removal as shown and described herein. As will be seen, in some instances, embodiments and/or methods of use of a punch assembly may bring about hair follicle removal with reduced healing time of a wound created at an extraction site. Such reduced healing time may be realized as a consequence of unique structural features of the punch assembly. Such features may include a toothed or denticulated distal portion, which, responsive to application of lateral forces during hair follicle extraction or removal operations, may give rise to a scallop-shaped inferior margin of a wound and an irregularly-shaped superior margin of the wound. The wound may comprise an overall ellipse shape which may heal in a reduced time span in comparison with circular-shaped wounds brought about by punch assemblies that merely transect surrounding tissues, thereby resulting in a circular-shaped wound.

In this context a “punch” or “punch assembly” refers to a hollow and/or substantially cylindrical device, which may be positioned along the axis of a hair follicle situated within a human scalp. The hollow and substantially cylindrical device may include a chamber, which may be suitable for encircling and receiving at least a portion of a hair follicle. Following encirclement of a hair follicle by the cylinder-shaped chamber, lateral and axial forces may be applied to the punch or punch assembly, which may bring about distortion of tissue surrounding the hair follicle. An applied axial force may give rise to a scallop-shaped separation at an inferior margin of the hair follicle with the surrounding tissue. At a superior margin, corresponding to a portion of the punch or punch assembly that lies in the direction opposite to the applied lateral force, an irregularly-shaped separation of the hair follicle from the surrounding tissue may be formed. Responsive to the elastic distortion or stretching of the tissue surrounding the hair follicle, an ellipse-shaped wound may result. Additionally, the ellipse-shaped wound may comprise a surface area that is reduced in size with respect to the surface area of the distal portion of the punch. Such an ellipse-shaped wound may facilitate reduced healing time and reduced scarring in relation to more typical, circular punches that operate to remove hair follicles by transection of tissue and without elastic distortion of tissue surrounding the hair follicle.

It should be noted that application of lateral force, in which the lateral force is oriented to align with Langer's lines on a human scalp, may further accelerate healing or mending of edges of a wound resulting from a hair follicle removal. In general an ellipse-shaped wound having greater eccentricity may heal faster than ellipse-shaped wounds having lesser eccentricity. In particular embodiments, ellipse-shaped wounds having eccentricities between about 0.5 and about 0.95 may bring about significantly faster healing than circle-shaped wounds, or wounds having eccentricities of less than 0.5. In certain embodiments, an ellipse-shaped wound having an eccentricity of between about 0.63 and about 0.8 may provide faster healing without unduly extending the length of the wound.

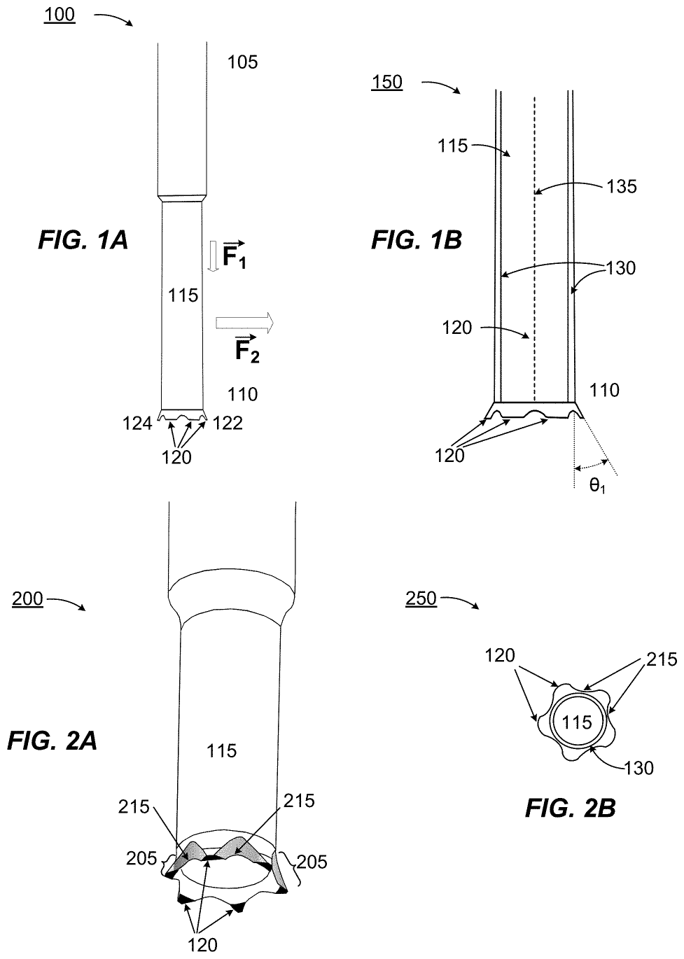

FIG. 1A is a side view illustrating various features of a punch assembly to facilitate hair follicle removal, according to an embodiment 100. As depicted in embodiment 100, a cylindrical punch assembly may comprise proximal end 105 and narrowed distal end 110. Distal end 110 may comprise a length in an axial direction of between about 1.75 mm and about 5.0 mm. Distal end 110 may comprise a width of between about 0.5 mm and about 0.75 mm. Distal end 110 may comprise a predominantly or substantially cylindrical receiving chamber 115, which may be suitable for receiving and retaining a hair follicle during a removal process. Denticulations 120 may resemble sharpened, tooth-like structures capable of incising tissue surrounding a hair follicle during hair removal process responsive to application of axial force F₁. In particular embodiments, denticulations 120 may comprise a sharpened lateral edge. Denticulations 120 located at side 122 of distal end 110, (which may correspond to an inferior margin) may bring about a scallop-shaped incision into tissue surrounding a hair follicle responsive to application of axial force F₁ and lateral force F₂. Also as shown in greater detail herein, denticulations 120 located at side 124 of distal end 110, (which may correspond to a superior margin) may bring about an irregularly-shaped wound, in response to denticulations 120 incising and/or tearing a hair follicle from surrounding tissue responsive to application of axial force F₁ and lateral force F₂. In particular embodiments, between 3 and 10 denticulations may be utilized at distal end 110. In certain embodiments, between 5 and 6 denticulations may be utilized at distal end 110.

A punch assembly, such as in accordance with FIG. 1A, may comprise one or more materials of a variety of materials, such as stainless steel, titanium, or any other metal alloy. Denticulations and or other areas of distal end 110 may be surface coated with hard thin films like titanium nitride. However, these are merely examples of punches, materials and methods of configuring a cutting edge and claimed subject matter is not limited in this respect.

FIG. 1B is a side view illustrating various features of a punch assembly to facilitate hair follicle removal, according to an embodiment 150. As depicted in embodiment 150, chamber walls 130 are oriented so as to form receiving chamber 115, which thereby permit a hair follicle to be situated within chamber 115. Denticulations 120 may be beveled at an angle Θ₁, so as to extend beyond the lateral boundaries of receiving chamber 115. In particular embodiments, lateral edges denticulations 120 may be sharpened so as to perform both and incising action as well as a tearing action during the surgical procedure, such as removal of a targeted hair follicle. In particular embodiments, angle Θ₁, which may comprise a bevel angle of denticulations 120 may comprise an angle of between about 15° and about 45° with respect to a direction parallel to central axis 135 of receiving chamber 115. In certain embodiments, angle Θ₁ may comprise an angle of between about 22° and about 38° with respect to a direction parallel to central axis 135 of receiving chamber 115. In the embodiment of FIG. 1B, in addition to incising and/or facilitating tearing of tissue surrounding a hair follicle, denticulations 120 may operate to assist in encircling a hair follicle in a manner that constrains the hair follicle within receiving chamber 115.

FIG. 2A is a view showing features of a distal portion of a punch assembly to facilitate hair follicle removal, according to an embodiment 200. As depicted in FIG. 2A, denticulations 120 may be separated by cutting edges 215, which may operate in concert with denticulations 120 to separate a targeted hair follicle from surrounding tissue. Beveled edges 205 may operate to guide a targeted hair follicle to the volume defined by receiving chamber 115.

FIG. 2B is a simplified top view showing features of a punch assembly to facilitate hair follicle removal, according to an embodiment 250. As shown in FIG. 2B, receiving chamber 115 may be surrounded, at least at a distal extremity of the punch assembly, by denticulations 120 interspersed by cutting edges 215. Denticulations 120 may be beveled, so as to be oriented away from the central axis of receiving chamber 115, while cutting edges 215 may be disposed more proximate with (or to coincide with) chamber walls 130 of the punch assembly depicted in FIG. 2B.

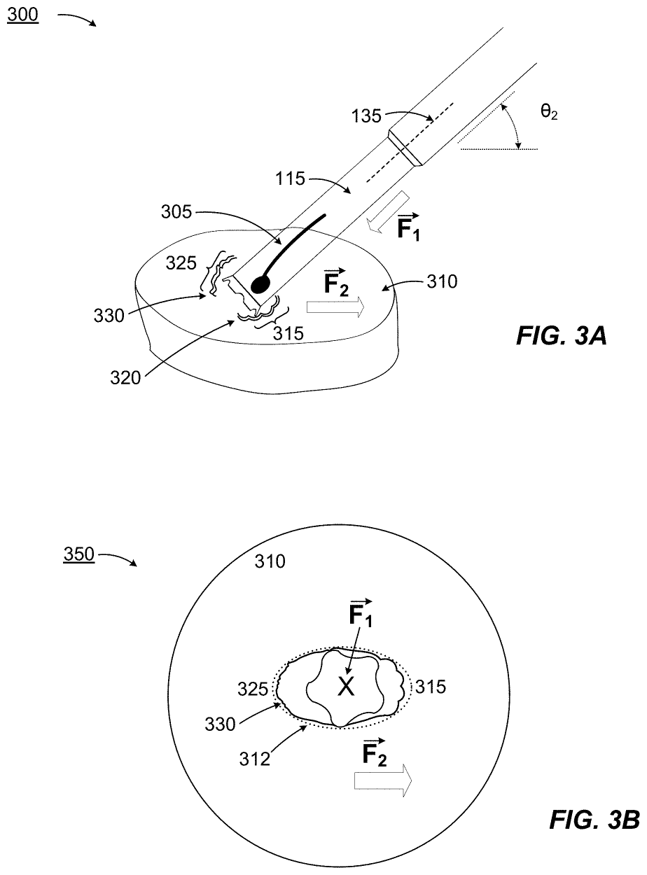

FIG. 3A is a diagram showing a punch assembly involved in a hair follicle removal process, according to an embodiment 300. As depicted in FIG. 3A a punch assembly may be oriented at an angle corresponding to Θ₂ so as to at least approximately align with hair follicle 305, shown as embedded within surrounding tissue 310. In the embodiment of FIG. 3, after encircling hair follicle 305 by receiving chamber 115, a first force (F₁) may be applied substantially in alignment with central axis 135. Responsive to application of force F₁, denticulations 120 may grip or hold into place a portion of surrounding tissue 310. The application of a second force (F₂) in a lateral direction, perhaps substantially along the plane of surrounding tissue 310, may bring about stretching or distortion of the surrounding tissue. As additional force F₁ is applied, denticulations 120 are pushed into surrounding tissue 310 at inferior margin 315, thereby forming a scallop-shaped separation of the tissue immediately surrounding hair follicle 305. The scallop-shaped separation at inferior margin 315 may be in response to denticulations 120 holding a portion of surrounding tissue 310 into place while cutting edges 215 form the scallop-shaped separation in the surrounding tissue. Responsive to additional force F₂ being applied in a lateral direction, an additional and irregularly-shaped separation may be formed at superior margin 325. Thus, as shown in FIG. 3A, a scallop-shaped separation may be formed at inferior margin 315, and an irregularly-shaped separation may be formed at superior margin 325. As previously noted, the resulting wound in surrounding tissue 310 having an irregularly-shaped at a superior margin and a scallop shape at an inferior margin may bring about an ellipse-shaped wound, which may heal faster than wounds of other shapes, such as circle-shaped wounds.

FIG. 3B is a top view showing a punch assembly involved in a hair follicle removal process, according to an embodiment 350. As shown in FIG. 3B, in response to force F₁ (shown as an “X” in FIG. 3B to indicate) a force directed into the page, and in response to lateral force F₂ being directed along the plane of surrounding tissue 310, scallop-shaped separation 320 may be formed at inferior margin 315. In addition, as force F₂ is applied, which may bring about formation of an irregularly-shaped tear or separation (330) at a superior margin of surrounding tissue 310. Application of forces F₁ and F₂ results in formation of an ellipse-shaped wound bounded by ellipse 312. Ellipse 312 is shown as bounding irregularly-shaped superior margin 325 and scallop-shaped inferior margin 315.

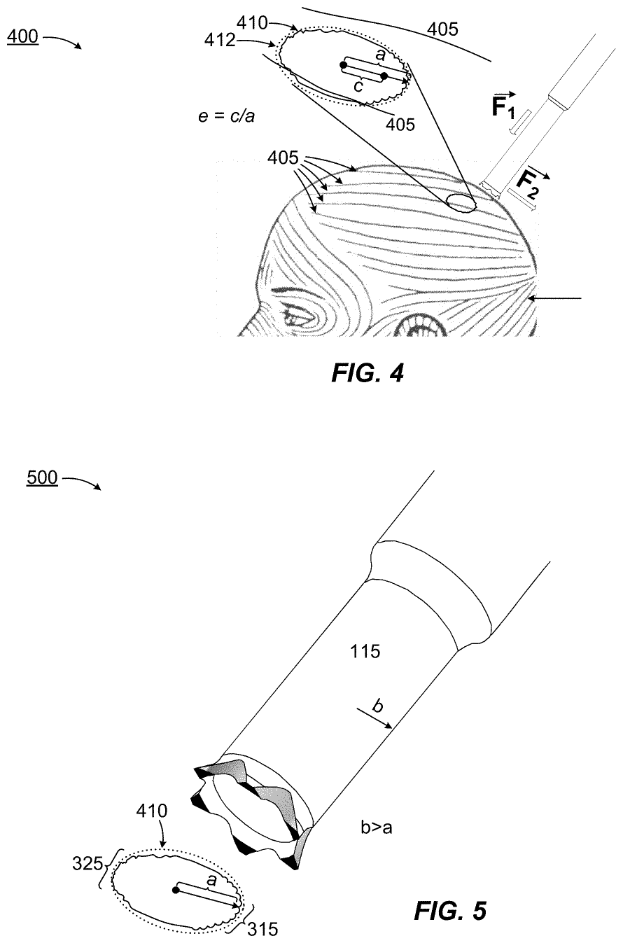

FIG. 4 is a diagram showing a wound imparted by a punch assembly to facilitate hair follicle removal on a human scalp relative to Langer's lines, according to an embodiment 400. Langer's lines, as shown in FIG. 4, represent lines of tension in human skin, which indicate the direction of orientation of underlying collagen fibers. Accordingly, if tissue is disrupted parallel to one or more of Langer's lines, separations in the tissue tend to re-approximate relatively quickly. Thus, as shown in FIG. 4, ellipse-shaped wound 410 may be formed in response to axial force F₁, which may be applied substantially perpendicular to a surface of the human scalp, while pulling (via application of lateral force F₂) in a direction that is tangential to the human scalp and parallel with one or more of Langer's lines 405. In FIG. 4, ellipse-shaped wound 410 is shown as being bounded by ellipse 412. FIG. 4 additionally indicates an irregularly-shaped portion of wound 410 at the superior margin of the wound, and a scallop-shaped portion at the inferior margin of the wound.

FIG. 5 is a diagram showing size of a wound imparted by a punch assembly to facilitate hair follicle removal relative to dimensions of the punch assembly, according to an embodiment 500. As depicted in FIG. 5, which may occur responsive to an axial force (F₁) and a lateral force F₂ being applied to a distal end of a punch assembly. As previously discussed herein, denticulations 120 (of FIG. 1A) may grip or hold into place a portion of the tissue surrounding a hair follicle while the surrounding tissue is distorted via application of a lateral force parallel to the surface of the surrounding tissue. Following formation of a wound and the surrounding tissue, in which the hair follicle may be removed from the surrounding tissue, the wound may return to a previous, undistorted shape, which may consume less surface area than distal end 110 of the punch assembly. FIG. 5 additionally indicates an irregularly-shaped portion of wound 410 at the superior margin of the wound, and a scallop-shaped portion at the inferior margin of the wound.

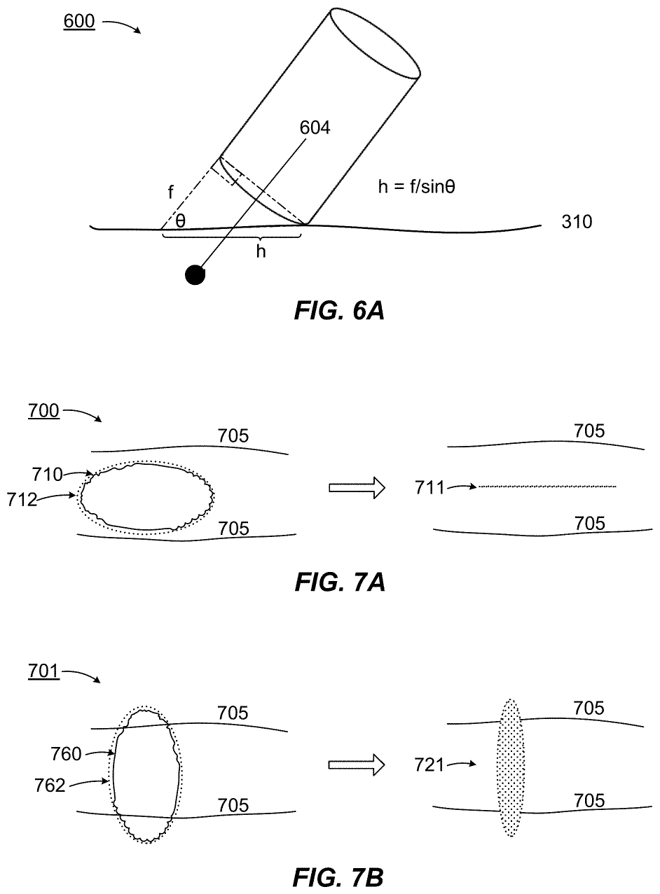

FIG. 6 shows a conventional cylindrical punch for hair follicle removal and illustrates disadvantages of such conventional cylindrical punches according to implementation 600. As shown in FIG. 6, a conventional cylindrical punch is situated in contact with surrounding tissue 310 so as to receive hair follicle 604. It may be appreciated that to accommodate hair follicle 604, the conventional cylindrical punch may be oriented at an angle Θ. It may also be appreciated that responsive to angle Θ decreasing, the portion of surrounding tissue 310 which must be incised by the conventional cylindrical punch increases as a function of the sine of angle Θ. The relationship between the length (h) of the portion of surrounding tissue 310 incised by the conventional cylindrical punch is thus given by expression (1) below:

$$h = \frac{f}{sin Θ}$$

Thus, as an example, for an angle Θ=60°, Table I (below) provides the incision length (h) of the portion of surrounding tissue severed by a conventional cylindrical punch of various diameters:

| Cylindrical Punch Diameter | Angle (Θ) | Incision Length (h) |
|---|---|---|
| 0.8 mm | 60° | 0.92 mm |
| 1.0 mm | 60° | 1.15 mm |
| 1.15 mm | 60° | 1.33 mm |
| 1.25 mm | 60° | 1.44 mm |

From Table I, it may be appreciated that for a conventional cylindrical punch, an incision length (h) is generally increased by a factor of approximately 15% with respect to the diameter of the punch (for Θ=60°). In another example, however, such as responsive to Θ=30°, an incision length (h) is generally increased by an even greater factor, such as 50% as indicated in expression (1).

In contrast to the implementation of FIG. 6, a denticulated punch assembly, such as the punch assembly to facilitate hair follicle removal according to embodiment 150 (of FIG. 1B), may give rise to an incision length (h) that is comparable, or even less than, an actual diameter of the punch assembly. Hair follicle removal procedures performed utilizing the denticulated punch assembly shown in the embodiments of FIGS. 1-5 bring about wounds that are horizontally-oriented ellipses. Responsive to wounds comprising a horizontal shape, there are no increases in diameter in the horizontal plane of a wound. In response to the vertical length of the wound being less than the diameter of the denticulated punch assembly, the wound may, in many instances, be smaller than the diameter of the punch. It may be appreciated that the overall design of the denticulated punch assembly allows the surgeon to cut deeper without increasing the risk of follicle damage. A deeper incision allows for easier removal of a graft following an incision.

FIG. 7A shows an ellipse-shaped wound in relation to Langer's lines (705), which may be brought about in response to a hair follicle removal procedure performed utilizing the denticulated punch assembly shown in FIGS. 1-5, according to an embodiment 700. As shown in FIG. 7A, ellipse-shaped wound 710 is depicted as being bounded by ellipse 712. After a healing process occurs, ellipse-shaped wound 710 is shown as collapsing into vertical line-shaped scar 711. Collapsing of an ellipse-shaped wound into a vertical line illustrates the advantage of the use of the denticulated punch assembly shown in FIGS. 1-5, which brings about reduced scarring, decreased healing time, reduced likelihood of infection, and so forth.

FIG. 7B shows an ellipse-shaped wound that intersects Langer's lines, according to an embodiment 701. In contrast to FIG. 7A responsive to ellipse-shaped wound 760 (bounded by ellipse 762) that intersects one or more of Langer's lines (705), a much more pronounced ellipse-shaped scar (721) is depicted. Ellipse-shaped scar 721 may represent a scar having much greater surface area than vertical line-shaped scar 711 of FIG. 7A.

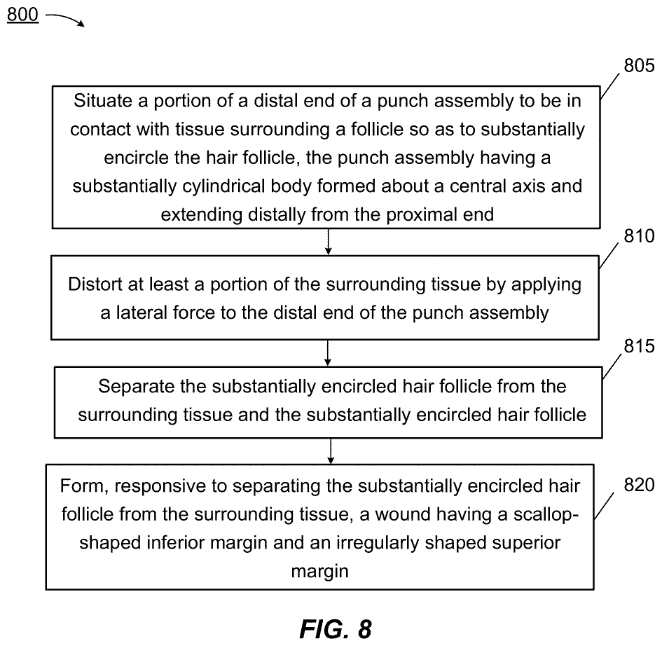

FIG. 8 is a flowchart for a method of using a punch assembly to facilitate hair follicle removal, according to an embodiment 800. The method may begin at 805, which may include situating a portion of a distal end of a punch assembly to be in contact with tissue surrounding a follicle, so as to substantially encircle the hair follicle. The punch assembly may include a substantially cylindrical body formed about a central axis and extended distally from a proximal end. The method may continue at 810, which may include distorting at least a portion of the surrounding tissue by applying a lateral force to the distal end of the punch assembly. At 815, the method may include separating the substantially encircled hair follicle from the surrounding tissue. At 820, the method may include forming, responsive to separating the substantially encircled hair follicle from the surrounding tissue, a wound having a scallop-shaped inferior margin and an irregularly-shaped superior margin.

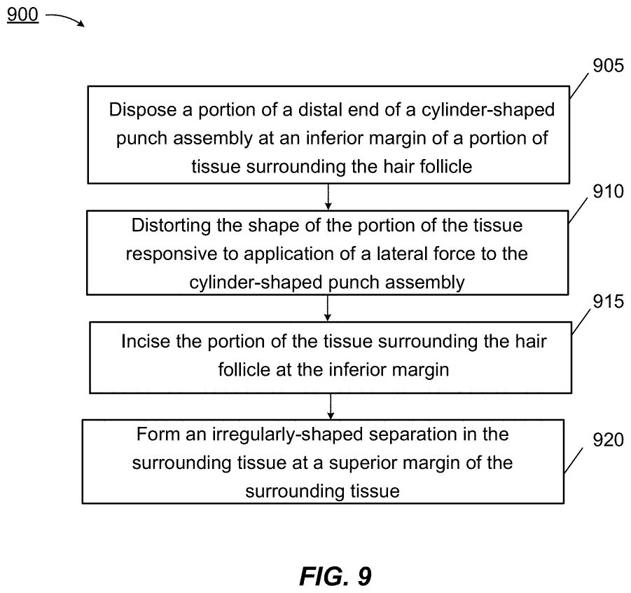

FIG. 9 is a flowchart for a method of using a punch assembly to facilitate hair follicle removal, according to an embodiment 900. The method may begin at 905, which may include disposing a portion of a distal end of a cylinder-shaped a punch assembly at an inferior margin of a portion of tissue surrounding the hair follicle. The method may continue at 910, which may include distorting the shape of the portion of the tissue responsive to application of a lateral force to the cylinder-shaped punch assembly. The method may continue at 915, which may include incising the portion of the tissue surrounding the hair follicle at the inferior margin. The method may continue at 920, which may include forming an irregularly-shaped separation in the surrounding tissue at a superior margin of the surrounding tissue.

Unless otherwise indicated, in the context of the present patent application, the term “or” if used to associate a list, such as A, B, or C, is intended to mean A, B, and C, here used in the inclusive sense, as well as A, B, or C, here used in the exclusive sense. With this understanding, “and” is used in the inclusive sense and intended to mean A, B, and C; whereas “and/or” can be used in an abundance of caution to make clear that all of the foregoing meanings are intended, although such usage is not required. In addition, the term “one or more” and/or similar terms is used to describe any feature, structure, characteristic, and/or the like in the singular, “and/or” is also used to describe a plurality and/or some other combination of features, structures, characteristics, and/or the like. Likewise, the term “based on” and/or similar terms are understood as not necessarily intending to convey an exhaustive list of factors, but to allow for existence of additional factors not necessarily expressly described.

Furthermore, it is intended, for a situation that relates to implementation of claimed subject matter and is subject to testing, measurement, and/or specification regarding degree, that the particular situation be understood in the following manner. As an example, in a given situation, assume a value of a physical property is to be measured. If alternatively reasonable approaches to testing, measurement, and/or specification regarding degree, at least with respect to the property, continuing with the example, is reasonably likely to occur to one of ordinary skill, at least for implementation purposes, claimed subject matter is intended to cover those alternatively reasonable approaches unless otherwise expressly indicated. As an example, if a plot of measurements over a region is produced and implementation of claimed subject matter refers to employing a measurement of slope over the region, but a variety of reasonable and alternative techniques to estimate the slope over that region exist, claimed subject matter is intended to cover those reasonable alternative techniques unless otherwise expressly indicated.

To the extent claimed subject matter is related to one or more particular measurements, such as with regard to physical manifestations capable of being measured physically, such as, without limit, temperature, pressure, voltage, current, electromagnetic radiation, etc., it is believed that claimed subject matter does not fall with the abstract idea judicial exception to statutory subject matter. Rather, it is asserted, that physical measurements are not mental steps and, likewise, are not abstract ideas.

It is noted, nonetheless, that a typical measurement model employed is that one or more measurements may respectively comprise a sum of at least two components. Thus, for a given measurement, for example, one component may comprise a deterministic component, which in an ideal sense, may comprise a physical value (e.g., sought via one or more measurements), often in the form of one or more forces, signal samples and/or states, and one component may comprise a random component, which may have a variety of sources that may be challenging to quantify. At times, for example, lack of measurement precision may affect a given measurement. Thus, for claimed subject matter, a statistical or stochastic model may be used in addition to a deterministic model as an approach to identification and/or prediction regarding one or more measurement values that may relate to claimed subject matter.

For example, a relatively large number of measurements may be collected to better estimate a deterministic component. Likewise, if measurements vary, which may typically occur, it may be that some portion of a variance may be explained as a deterministic component, while some portion of a variance may be explained as a random component. Typically, it is desirable to have stochastic variance associated with measurements be relatively small, if feasible. That is, typically, it may be preferable to be able to account for a reasonable portion of measurement variation in a deterministic manner, rather than a stochastic matter as an aid to identification and/or predictability.

Along these lines, a variety of techniques have come into use so that one or more measurements may be processed to better estimate an underlying deterministic component, as well as to estimate potentially random components. These techniques, of course, may vary with details surrounding a given situation. Typically, however, more complex problems may involve use of more complex techniques. In this regard, as alluded to above, one or more measurements of physical manifestations may be modelled deterministically and/or stochastically. Employing a model permits collected measurements to potentially be identified and/or processed, and/or potentially permits estimation and/or prediction of an underlying deterministic component, for example, with respect to later measurements to be taken. A given estimate may not be a perfect estimate; however, in general, it is expected that on average one or more estimates may better reflect an underlying deterministic component, for example, if random components that may be included in one or more obtained measurements, are considered. Practically speaking, of course, it is desirable to be able to generate, such as through estimation approaches, a physically meaningful model of processes affecting measurements to be taken.

In the preceding description, various aspects of claimed subject matter have been described. For purposes of explanation, specifics, such as amounts, systems and/or configurations, as examples, were set forth. In other instances, well-known features were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all modifications and/or changes as fall within claimed subject matter.

What is claimed is:

1. A punch assembly for removing hair follicles from surrounding tissue, the punch assembly comprising:
a substantially cylindrical body having a proximal end and a distal end, the substantially cylindrical body formed about a central axis and extending distally from the proximal end, the substantially cylindrical body having a hair follicle-receiving chamber at least partially defined by an interior wall within the substantially cylindrical body, the interior wall extending distally from the proximal end; and
a plurality of denticulations arranged at the distal end of the substantially cylindrical body, the plurality of denticulations being beveled in a direction away from the central axis so as to form an outer cutting edge.

2. The punch assembly of claim 1, wherein the plurality of denticulations comprises between 3 and 10 denticulations.

3. The punch assembly of claim 1, wherein the plurality of denticulations consists of 5 or 6 denticulations.

4. The punch assembly of claim 1, wherein the substantially cylindrical body comprises a narrowed portion that terminates at the distal end, the narrowed portion having a length of between 1.75 mm and 5.0 mm.

5. The punch assembly of claim 4, wherein the narrowed portion has a width of between 0.5 mm and 0.75 mm.

6. The punch assembly of claim 1, wherein the plurality of denticulations are beveled at an angle of between about 15° and about 45° with respect to the central axis.

7. The punch assembly of claim 6, wherein the plurality of denticulations are beveled at an angle of between about 22° and about 38° with respect to the central axis.

8. A method of removing a hair follicle from surrounding tissue for use with a punch assembly having a distal end and a proximal end, the method comprising:
situating a portion of the distal end of the punch assembly to be in contact with the surrounding tissue so as to substantially encircle the hair follicle, the punch assembly having a substantially cylindrical body formed about a central axis and extending distally from the proximal end;
distorting at least a portion of the surrounding tissue by applying a lateral force to the distal end of the punch assembly;
separating the substantially encircled hair follicle from the surrounding; and
forming, responsive to separating the substantially encircled hair follicle from the surrounding tissue, a wound having a scalloped-shaped inferior margin and an irregularly shaped superior margin.

9. The method of claim 8, wherein separating the substantially encircled hair follicle from the surrounding tissue comprises tearing the substantially encircled hair follicle from the surrounding tissue.

10. The method of claim 8, further comprising:
incising into the surrounding tissue and in a direction substantially parallel to the hair follicle while maintaining the situated portion of the distal end of the punch assembly in contact with the surrounding tissue.

11. The method of claim 10, further comprising:
maintaining the lateral force to the distal end of the punch assembly while incising into the surrounding tissue.

12. The method of claim 11 further comprising:
extracting the hair follicle in a manner that forms a wound in the surrounding tissue having an ellipse shape with a measure of eccentricity of between about 0.5 and about 0.95.

13. The method of claim 12, wherein the ellipse shape comprises a measure of eccentricity of between about 0.63 and about 0.8.

14. The method of claim 12, wherein the ellipse-shaped wound comprises a major axis that substantially aligns with one or more of Langer's lines proximate the wound.

15. The method of claim 8, wherein distorting the portion of the surrounding tissue reduces an axial force to initiate an incision into the surrounding tissue by at least about 25%.

16. A method of removing a hair follicle, comprising:
disposing a portion of a distal end of a cylinder-shaped punch assembly at an inferior margin of a portion of tissue surrounding the hair follicle;
distorting the shape of the portion of the tissue responsive to application of a lateral force to the cylinder-shaped punch assembly;
incising the portion of the tissue surrounding the hair follicle at the inferior margin; and
forming an irregularly-shaped separation in the surrounding tissue at a superior margin of the surrounding tissue.

17. The method of claim 16, wherein distorting the shape of the portion of the surrounding tissue comprises:
reducing axial force to bring about the incision of the portion of the surrounding tissue at the inferior margin.

18. The method of claim 16, further comprising:
extracting the hair follicle responsive to applying force that at least approximately aligns with the central axis of the hair follicle.

19. The method of claim 18, further comprising:
producing an ellipse-shaped wound responsive to extracting the hair follicle and removing the cylinder-shaped punch assembly from the portion of the surrounding tissue.

20. The method of claim 19, wherein the ellipse-shaped wound comprises a major access that substantially aligns with one or more of Langer's lines proximate the wound.
